(19) United States
(12) Patent Application Publication
(10) Pub. No.: US 2019/0008466 A1
(43) Pub. Date: Jan. 10, 2019

(54) LIFE LOG UTILIZATION SYSTEM, LIFE LOG UTILIZATION METHOD, AND RECORDING MEDIUM

(71) Applicant: HONDA MOTOR CO., LTD., Tokyo (JP)

(72) Inventors: Miyuki Shimota, Wako-shi (JP); Takaichi Sano, Wako-shi (JP); Makoto Yuzawa, Wako-shi (JP); Atsushi Nishi, Wako-shi (JP); Keiichi Iguchi, Tokyo (JP); Seiichi Yamamoto, Tokyo (JP)

(21) Appl. No.: 16/026,379

(22) Filed: Jul. 3, 2018

(30) Foreign Application Priority Data
Jul. 10, 2017 (JP) 2017-134661

(51) Int. Cl. A61B 5/00; G06K 9/00; A61B 5/16; G06T 19/00; G02B 27/01

(52) U.S. Cl. CPC A61B 5/7289; G06K 9/00335; A61B 5/165; A61B 5/02405; G06T 19/006; G02B 27/0172; A61B 5/167

(57) ABSTRACT

A life log utilization system associates event information, emotion information, and behavior information, which are acquired at the same time or within a predetermined time period, records the associated information as a life log of a target person or a user, and outputs the life log or information, such as analysis data and analysis content, generated from the life log as personality analysis information.

### CROSS-REFERENCE TO RELATED APPLICATION

This application is based upon and claims the benefit of priority from Japanese Patent Application No. 2017-134661 filed on Jul. 10, 2017, the contents of which are incorporated herein by reference.

### BACKGROUND OF THE INVENTION

### Field of the Invention

The present invention relates to a life log utilization system, a life log utilization method, and a recording medium, which make use of a life log collected from a target person.

### Description of the Related Art

In recent years, along with the rapid development of information processing technology, services for enriching people's lives are being provided through the analysis of so-called big data. For example, various techniques have been developed for utilizing a life log collected from a target person.

In Japanese Laid-Open Patent Publication No. 2014-191731, an event recording device has been proposed in which the occurrence of an event acts as a trigger in order to count the number of detection times of the event caused by operation of a portable device, and the number of detection times is recorded and displayed as an event that imparts an influence on the emotions of a target person. As a specific example, a statement may be provided to the effect that the device has detected the number of times of striking a button, the number of vibrations, the number of times that pressure is applied to the device, or the number of times that the temperature of the device changes.

### SUMMARY OF THE INVENTION

However, with the apparatus proposed in Japanese Laid-Open Patent Publication No. 2014-191731, information is simply indicated concerning an event that has an influence on the emotions, and a relationship of behaviors and emotions of the target person with respect to the occurrence of the event cannot be grasped. Stated otherwise, when analyzing the personality of the target person objectively and in detail, there is a problem in that it is impossible to obtain sufficient useful information.

The present invention has been devised with the aim of solving the aforementioned problem, and has the object of providing a life log utilization system, a life log utilization method, and a program, which are capable of presenting useful information to a target person in relation to the individual characteristics and personality of the target person.

A life log utilization system according to one aspect of the present invention includes an event information acquisition unit configured to acquire event information in relation to an event occurring in the vicinity of a target person or in relation to an event involving the target person, an emotion information acquisition unit configured to acquire emotion information indicative of an emotional state of the target person, a behavior information acquisition unit configured to acquire behavior information indicative of a behavior of the target person, a life log recording unit configured to associate the event information acquired by the event information acquisition unit, the emotion information acquired by the emotion information acquisition unit, and the behavior information acquired by the behavior information acquisition unit, which are acquired at a same time or within a predetermined time period, and to record the associated information as a life log of the target person, and an analysis information output unit configured to output the life log recorded by the life log recording unit or information generated from the life log as personality analysis information.

In the foregoing manner, the event information, the emotion information, and the behavior information are associated when the life log is recorded, and such a life log or information generated from the life log is output. Therefore, from the standpoint of psychological behavior, it is possible to present useful information related to the personality of the target person, to the target person.

The life log utilization system may further include a biometric information acquisition unit configured to acquire biometric information indicative of biological activity of the target person, and the emotion information acquisition unit may acquire the emotion information on the basis of the biometric information acquired by the biometric information acquisition unit.

The life log utilization system may further include a life log specifying unit configured to specify, as a featured log, the life log for which a change in the emotion information thereof is equal to or greater than a threshold value, and the analysis information output unit may output the personality analysis information in relation to the featured log specified by the life log specifying unit. In accordance with this feature, it is possible to detect an unconscious awareness of the target person through a change in the emotional state of the target person, and to appropriately extract a featured log having high value as a target for analysis, from among a large number of life logs.

The life log utilization system may further include a life log specifying unit configured to specify, as a featured log, the life log for which a parameter in the life log or an indicator quantified or qualified using the parameter is specific, from among the life logs recorded in the past by the life log recording unit, and the analysis information output unit may output the personality analysis information in relation to the featured log specified by the life log specifying unit. In accordance with this feature, it is possible to detect an unconscious awareness of the target person through a specific parameter or indicator sought out from among a past history, and to appropriately extract a featured log having high value as a target for analysis, from among a large number of life logs.

The life log utilization system may further include a characteristic estimating unit configured to estimate a characteristic of the target person by extracting a commonality in the event information or the behavior information included in a plurality of the featured logs. In accordance with this feature, it is possible to express characteristics of the target person that were performed unconsciously before.

The life log utilization system may further include a related information acquisition unit configured to acquire related information, which is made up of the behavior information or the event information having a high degree of relevance to the characteristic estimated by the characteristic estimating unit, and the analysis information output unit may extract, from among the related information acquired by the related information acquisition unit, information that is related to a behavior or an event that the target person has not yet experienced, and output the extracted information at a predetermined timing to the target person. In accordance with this feature, it becomes possible to experience a behavior or an event in a pseudo manner by way of the related information, which enables the target person to have a new awareness of himself.

Further, the analysis information output unit may be configured to generate a virtual world in which a behavior of the target person is virtually reproduced using at least the featured log. In accordance with this feature, it is possible to reproduce experiences of the target person through the virtual world, and to enable the target person to look back from an objective standpoint on actions taken by the target person himself.

A life log utilization method according to another aspect of the present invention is configured to be executed by one or a plurality of computers, and includes an event information acquisition step of acquiring event information in relation to an event occurring in the vicinity of a target person or in relation to an event involving the target person, an emotion information acquisition step of acquiring emotion information indicative of an emotional state of the target person, a behavior information acquisition step of acquiring behavior information indicative of a behavior of the target person, a recording step of associating the event information, the emotion information, and the behavior information, which are acquired at the same time or within a predetermined time period, and recording the associated information as a life log of the target person, and an outputting step of outputting the recorded life log or information generated from the life log as personality analysis information.

In a non-transitory recording medium according to yet another aspect of the present invention, there is recorded a life log utilization program configured to cause one or a plurality of computers to perform an event information acquisition step of acquiring event information in relation to an event occurring in the vicinity of a target person or in relation to an event involving the target person, an emotion information acquisition step of acquiring emotion information indicative of an emotional state of the target person, a behavior information acquisition step of acquiring behavior information indicative of a behavior of the target person, a recording step of associating the event information, the emotion information, and the behavior information, which are acquired at the same time or within a predetermined time period, and recording the associated information as a life log of the target person, and an outputting step of outputting the recorded life log or information generated from the life log as personality analysis information.

In accordance with the life log utilization system, the life log utilization method, and the recording medium of the present invention, it is possible to present useful information in relation to the personality of a target person, to the target person.

The above and other objects, features, and advantages of the present invention will become more apparent from the following description when taken in conjunction with the accompanying drawings, in which a preferred embodiment of the present invention is shown by way of illustrative example.

### BRIEF DESCRIPTION OF THE DRAWINGS

FIG. 1 is an overall configuration diagram of a life log utilization system according to an embodiment of the present invention;

FIG. 2 is a functional block diagram of a personality analysis support server shown in FIG. 1;

FIG. 3 is a first flowchart for describing operations of the life log utilization system shown in FIG. 1;

FIG. 4 is a second flowchart for describing operations of the life log utilization system shown in FIG. 1;

FIG. 5A is a diagram illustrating an example of a data structure of the life log;

FIG. 5B is a diagram illustrating an example of a data structure of analysis data;

FIG. 6 is a diagram schematically illustrating a first method of specifying a life log;

FIG. 7 is a diagram schematically illustrating a second method of specifying a life log;

FIG. 8 is a third flowchart for describing operations of the life log utilization system shown in FIG. 1;

FIG. 9 is a diagram showing an example of an output form of analysis content;

FIG. 10 is a fourth flowchart for describing operations of the life log utilization system shown in FIG. 1; and

FIG. 11 is a diagram illustrating an example of a data structure of user characteristic data.

### DESCRIPTION OF THE PREFERRED EMBODIMENTS

A preferred embodiment of a life log utilization system according to the present invention, in relation to a life log utilization method and a life log utilization program, will be presented and described in detail below with reference to the accompanying drawings.

### [Configuration of Life Log Utilization System 10]

### <Overall Configuration>

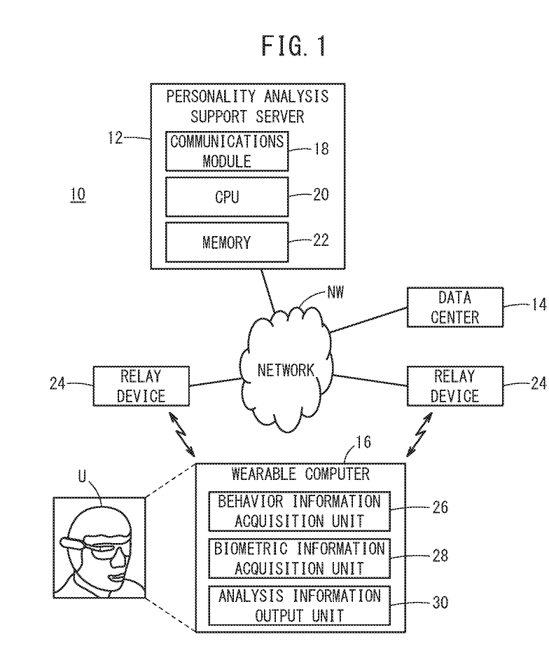

FIG. 1 is an overall configuration diagram of a life log utilization system 10 according to an embodiment of the present invention. The life log utilization system 10 is a system configured to collect life logs for each of users U (target subjects) who have been registered as users of the service, and to analyze the personalities of the users U. More specifically, the life log utilization system 10 is configured to include a personality analysis support server 12, a data center 14, and a wearable computer 16.

The personality analysis support server 12 is a computer that plays a central role in the life log utilization system 10. The personality analysis support server 12 is configured to include a communications module 18, a CPU 20 (Central Processing Unit), and a memory 22. Moreover, the memory 22 is a non-transitory storage medium (non-transitory recording medium), and is constituted by a computer-readable storage medium.

A plurality of different types of databases (to be described later) are constructed by a server (not shown) installed in the data center 14. The data center 14 is configured to be capable of communicating mutually with the personality analysis support server 12 via a network NW (for example, the Internet). Consequently, data can be exchanged between the data center 14 and the personality analysis support server 12.

Incidentally, the personality analysis support server 12 and a plurality of relay devices 24 are mutually connected via the network NW. Consequently, the personality analysis support server 12 is capable of receiving data from the wearable computer 16 via the relay devices 24 and the network NW.

The wearable computer 16 is a multifunction/multipurpose device which can be used in a state of being worn by the user U, and is configured to include, for example, a housing, a control circuit board, a display panel, a speaker, or a plurality of sensors. Consequently, the wearable computer 16 functions as a behavior information acquisition unit 26, a biometric information acquisition unit 28, and an analysis information output unit 30.

The sensors that are mounted in the wearable computer 16 may include, for example, any of a camera, a vitality sensor, a motion sensor, a position sensor, or an audio sensor. More specifically, by analyzing sensor values from these sensors, it is possible to detect a heartbeat, a pulse, a blood pressure, the pupils, a line of sight, movements, a position, or a voice of the user U.

In the example shown in the drawings, the wearable computer 16 is made up of so-called VR (Virtual Reality) goggles, however, the wearable computer 16 may be a head mounted display having another form. Further, the wearable computer 16 may be configured to be capable of communicating with an attached output device (for example, a display, a projector), and instead of displaying images by itself, may display images on the output device.

### <Functional Block Diagram of Personality Analysis Support Server 12>

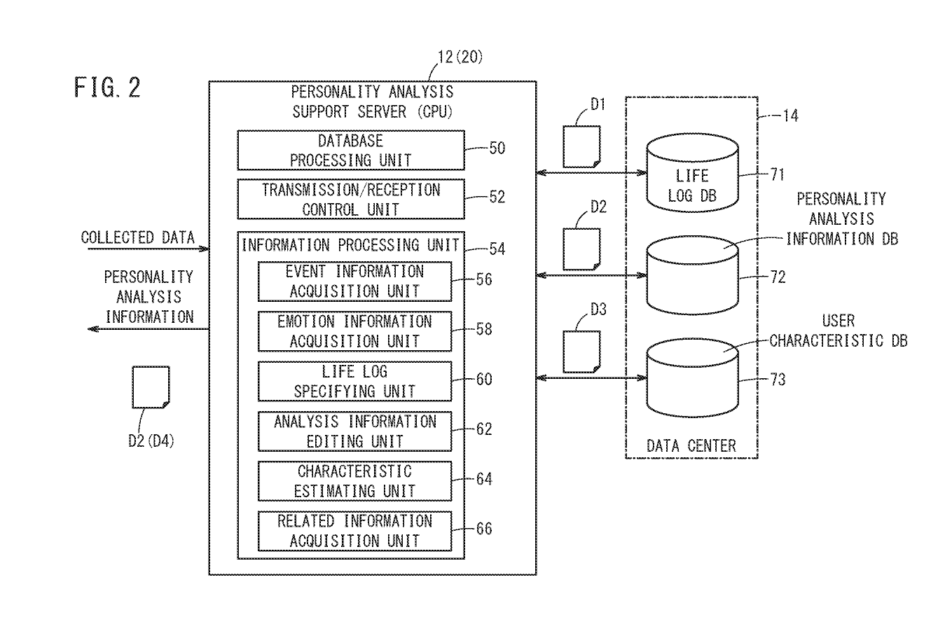

FIG. 2 is a functional block diagram of the personality analysis support server 12 shown in FIG. 1.

The CPU 20 of the personality analysis support server 12, by reading out and executing programs stored in the memory 22, functions as a database processing unit 50, a transmission/reception control unit 52, and an information processing unit 54. The information processing unit 54 is constituted to include an event information acquisition unit 56, an emotion information acquisition unit 58, a life log specifying unit 60, an analysis information editing unit 62, a characteristic estimating unit 64, and a related information acquisition unit 66.

In the data center 14, for example, there are constructed three types of databases, and more specifically, a database relating to a life log D1 (hereinafter referred to as a life log DB 71), a database relating to personality analysis data D2 (hereinafter referred to as a personality analysis information DB 72), and a database relating to user characteristic data D3 (hereinafter referred to as a user characteristic DB 73).

### [Operations of Life Log Utilization System 10]

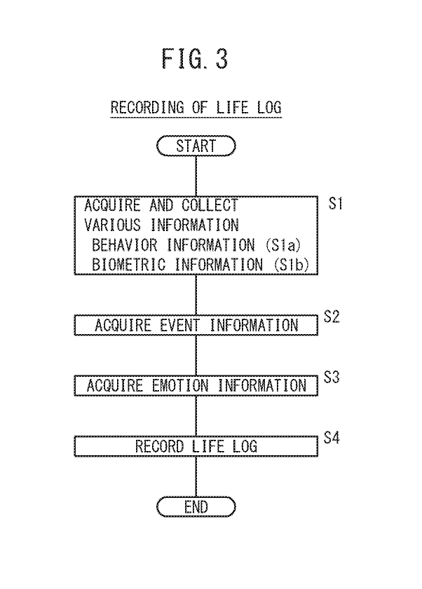

In the present embodiment, the life log utilization system 10 is configured in the manner described above. Next, a description will be presented with reference to FIGS. 3 to 10 concerning operations of the life log utilization system 10.

### <First Operation: Recording of Life Log D1>

In step S1 of FIG. 3, the life log utilization system 10 acquires various types of information used for generating the life log D1. More specifically, in step S1a, the behavior information acquisition unit 26 acquires information (hereinafter referred to as behavior information) indicative of behaviors of the user U. In step S1b, the biometric information acquisition unit 28 acquires information (hereinafter referred to as biometric information) indicative of biological activity of the user U.

Steps S1a and S1b may be executed synchronously, or may be executed asynchronously (for example, at each of different cycles). With an accumulation of a predetermined amount of data acting as a trigger, the wearable computer 16 transmits data (hereinafter referred to as operator data) including the behavior information and the biometric information, and which are collected from the user U, to the personality analysis support server 12.

After having received the data collected from each of the users U via the relay devices 24, the network NW, and the communications module 18, the personality analysis support server 12 temporarily stores the collected data in the memory 22.

In step S2, the event information acquisition unit 56 acquires event information associated with the information (particularly, the behavior information) that was acquired in step S1. More specifically, the event information acquisition unit 56 acquires the event information by extracting a portion of the behavior information, or by analyzing the content of the behavior information, and estimating an event.

In this instance, the “event information” is defined as information related to an event that occurs in the vicinity of the user U or an event involving the user U. Such an event may be a behavior unit that is classified to an extent for which behavior analysis thereof is possible, may be an abstract event (for example, a date or a festival), or may be a particular and concrete event (for example, a meeting, a conversation, a behavior taking place during commuting).

In step S3, the emotion information acquisition unit 58 acquires emotion information associated with the information (i.e., the collected data) that was acquired in step S1. More specifically, the emotion information acquisition unit 58 estimates the emotional state of the user U on the basis of the biometric information included in the collected data, and acquires an estimation result (quantitative value) as emotion information.

Prior to this estimation, for example, the emotion information acquisition unit 58 may detect a level of emotional excitement by grasping a change in the heart rate, or may detect a facial expression by carrying out image processing with respect to a camera image including the face. In addition, on the basis of one or a plurality of the aforementioned detection results, the emotion information acquisition unit 58 quantifies the emotional state of the user U using one or a plurality of parameters (for example, happiness, anger, sorrow, pleasure).

In this instance, although the emotion information acquisition unit 58 generates the emotion information on the basis of the biometric information of the user U, the method of acquiring the emotion information is not limited to this feature. For example, the emotion information acquisition unit 58 may newly acquire emotion information of the user U which is associated with the collected data by some method, from an external device that differs from the personality analysis support server 12.

In step S4, the data center 14 (and more specifically, the life log DB 71) associates the information acquired in steps S1 through S3, and records the associated information as a life log D1 of the user U. Prior to recording such information, the database processing unit 50 performs a process (i.e., a data coupling process) of associating information that was acquired at the same time or within a predetermined time period.

First, in the event that the acquisition time of behavior information coincides with the acquisition time of biometric information within an allowable range (for example, within one minute), the database processing unit 50 regards the behavior information and the biometric information as having relevance, and associates the behavior information with the biometric information. Thereafter, in the case that a predetermined condition for linking or associating the event information is satisfied, the database processing unit 50 further associates the event information with respect to the previously associated information set (the behavior information and the biometric information).

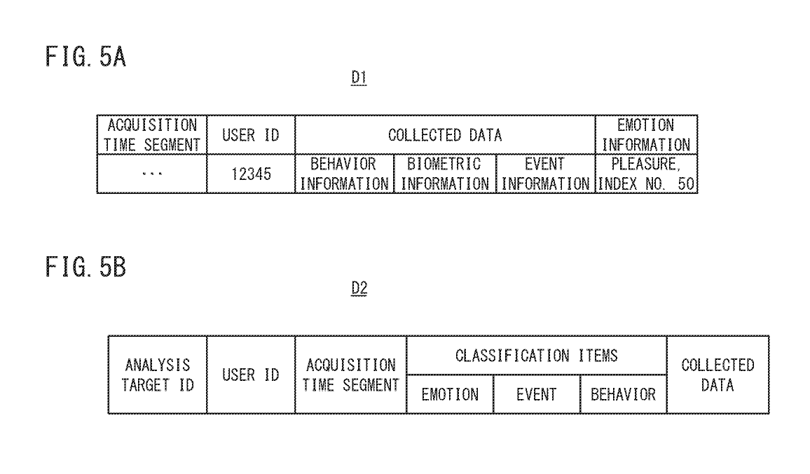

FIG. 5A is a diagram illustrating an example of a data structure of the life log D1. The present diagram corresponds to a record that makes up a constituent unit of the life log DB 71. The life log D1 is configured to include an acquisition time segment, a user ID, collected data (including behavior information, biometric information, and event information), and emotion information. In this instance, the user ID (=12345) is an identifier unique to the user U.

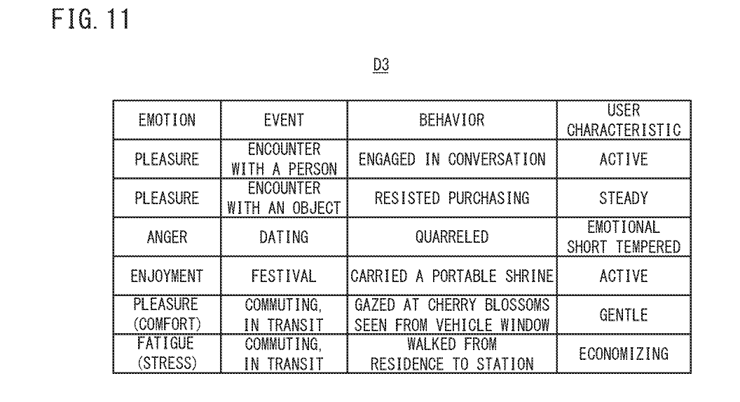

As long as it is possible to generate the record of the life log D1 shown in FIG. 5A, for example, an arbitrary type of database including a hierarchical type, a network type, or a relational type of database may be adopted. Such a feature also applies to the analysis data D2 (FIG. 5B) and the user characteristic data D3 (FIG. 11) which will be described later.

Thereafter, the transmission/reception control unit 52 transmits the data to be recorded including the life log D1 to the data center 14. Consequently, the life log DB 71 records the life log D1 of the user U which is formed by associating at least the behavior information, the biometric information, the event information, and the emotion information.

In this manner, the life log utilization system 10 completes the first operation (recording the life log D1). By repeatedly executing the process of the flowchart of FIG. 3 in a periodic or irregular manner, the life log utilization system 10 is capable of sequentially collecting life logs D1 of users U who have registered to use the service.

### <Second Operation: Generation of Analysis Data D2>

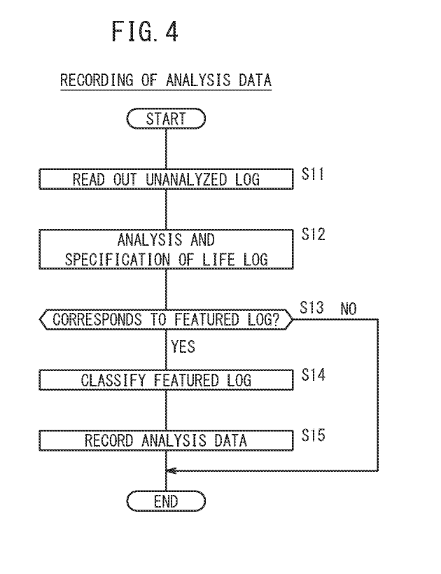

In step S11 of FIG. 4, the database processing unit 50 refers to the life log DB 71 of the data center 14, and reads out a life log D1 (an arbitrary number of records) which has not yet been analyzed. Such an analysis state is determined, for example, by referring to tag information of the life log D1 (for example, a flag indicative of the analysis state).

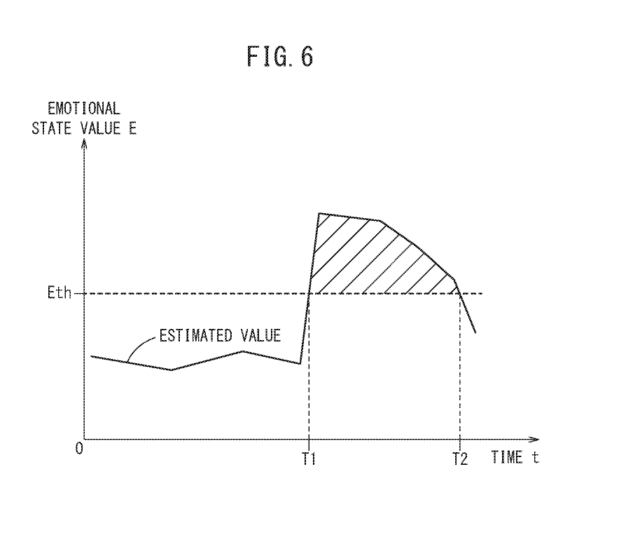

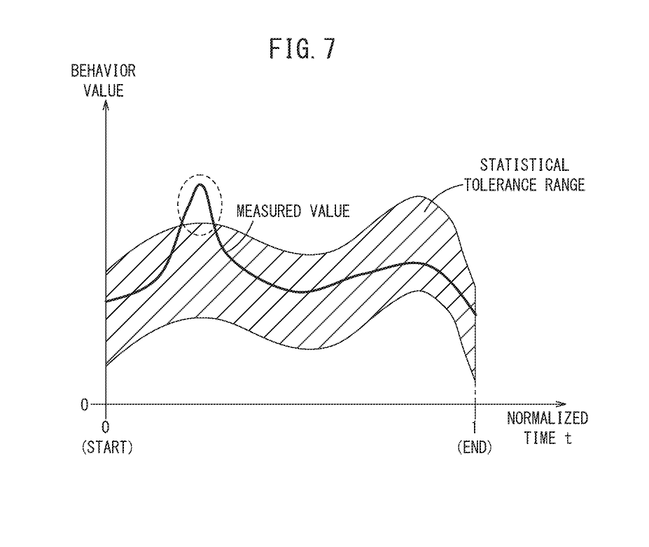

In step S12, the life log specifying unit 60 analyzes the content of the life log D1 read in step S11, and specifies whether or not it is a life log having high value as a target for analysis (hereinafter referred to as a featured log). Hereinafter, a specific example of such a specifying method will be described with reference to FIGS. 6 and 7.

FIG. 6 is a diagram schematically illustrating a first method of specifying a life log D1. The horizontal axis of the graph indicates an acquisition time (hereinafter simply referred to as a “time t”) of the biometric information, and the vertical axis of the graph indicates an emotional state value E (units: arbitrary). The state value E is a parameter indicative of the degree of emotional excitement. In the case that the value of E is large, a state of emotional excitement is indicated, whereas in the case that the value of E is small, a normal emotional state is indicated.

In this instance, the life log specifying unit 60 compares a magnitude relationship between a change over time of the state value E and a preset threshold value Eth. As a result, the state value E (estimated value) is regarded as satisfying the relationship of E≥Eth inside the hatched region (stated otherwise, within a time range of T1≤t≤T2). In this case, the life log specifying unit 60 specifies a life log D1 whose value (t) of the “acquisition time” lies within the range of T1≤t≤T2.

In this manner, the life log specifying unit 60 may specify, as a featured log, a life log D1 for which a change in the emotion information is greater than or equal to a threshold value. In accordance with this feature, it is possible to detect an unconscious awareness of the user U through a change in the emotional state of the user U, and to appropriately extract a featured log having high value as a target for analysis, from among a large number of life logs D1.

FIG. 7 is a diagram schematically illustrating a second method of specifying a life log D1. The horizontal axis of the graph indicates a time that is normalized to a range of [0, 1] (hereinafter referred to simply as a “normalized time t”), and the vertical axis of the graph indicates an amount of behavior (units: arbitrary). In the case that the value of the behavior amount is large, the user U is considered to be behaving in a large manner, whereas in the case that the value of the behavior amount is small, the user U is considered to be behaving in a small manner.

In this instance, the life log specifying unit 60 determines whether or not the change over time of the behavior information lies within a statistical tolerance range. As a result, it is assumed that the behavior amount (measured value) deviates from the statistical tolerance range in the time range encircled by the dashed line. In this case, the life log specifying unit 60 specifies a life log D1 lying within the range of the entire segment [0, 1].

The statistical tolerance range corresponds to a range in which a degree of statistical deviation of the behavior information is less than or equal to a threshold value, with past life logs D1 associated mutually with matching or similar events serving as a population. More specifically, the statistical tolerance range is formed by adding a margin amount which is equivalent in positive and negative directions with respect to an average function of the life logs D1.

In this manner, the life log specifying unit 60 specifies, as a featured log, a life log D1, from among the life logs D1 recorded in the past by the life log DB 71, for which a parameter in the life log D1, or an indicator quantified or qualified using the parameter is specific. In accordance with this feature, it is possible to detect an unconscious awareness of the user U through a specific parameter or indicator sought out from among a past history, and to appropriately extract a featured log having high value as a target for analysis, from among a large number of life logs D1.

In step S13, the life log specifying unit 60 determines whether the life log D1 that was analyzed in step S2 corresponds to a featured log. More specifically, in the case it is determined that a featured log does not exist among the targets for analysis (step S13: NO), the process of the flowchart is immediately brought to an end. On the other hand, in the case it is determined that a featured log does exist among the targets for analysis (step S13: YES), the process proceeds to the following step S14.

In step S14, the life log specifying unit 60 classifies the featured log that was specified in step S13, in accordance with a predetermined rule, and assigns the classification items concerning an “emotion”, an “event”, or a “behavior” to the featured log.

In step S15, the data center 14 (and more specifically, the personality analysis information DB 72) records the featured log that was specified in step S13 as analysis data D2 in which there is included the classification items assigned in step S14. Prior to recording the same, the database processing unit 50 performs a process (i.e., a data coupling process) of associating the featured log with the classification items.

FIG. 5B is a diagram illustrating an example of a data structure of the analysis data D2. The present diagram corresponds to a record that makes up a constituent unit of the personality analysis information DB 72. The analysis data D2 is configured to include an analysis target ID, a user ID, an acquisition time segment, classification items (an emotion, an event, a behavior), and the collected data. In this instance, the analysis target ID is an identifier that is unique to the analysis data D2, which is managed in a unitary manner in the personality analysis information DB 72.

Thereafter, the transmission/reception control unit 52 transmits the data to be recorded including the analysis data D2 to the data center 14. Consequently, the personality analysis information DB 72 records the analysis data D2 that characterizes the personality of the user U.

In the foregoing manner, the life log utilization system 10 completes the second operation (generation of the analysis data D2). The life log utilization system 10 is capable of generating the analysis data D2 from a large number of life logs D1, by repeatedly executing the process of the flowchart of FIG. 4, for example, within a time zone in which the operation rate of the personality analysis support server 12 is low.

### <Third Operation: Output of Analysis Content D4 (First Example)>

Incidentally, after the user U has experienced a specific event, there are cases in which the user U desires to objectively look back on his or her own actions. In this case, the life log utilization system 10 is capable of providing the analysis content D4 to the user U.

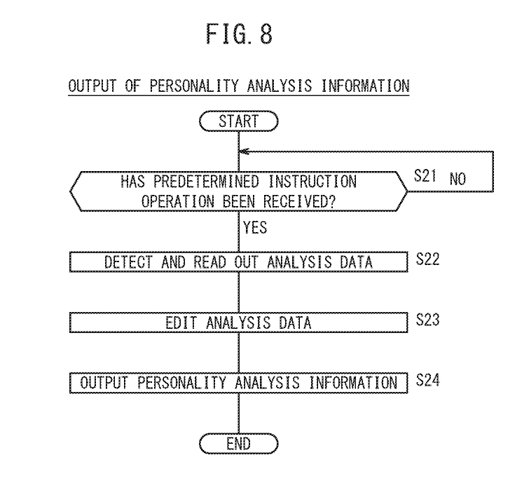

In step S21 of FIG. 8, the personality analysis support server 12 determines whether or not a predetermined instruction operation (i.e., a request or an acknowledgment from the user U) has been received from an external device (for example, the wearable computer 16). An interface (HMI: Human Machine Interface) for accepting such an instruction operation may adopt various forms, including a mode of selecting from a displayed content list.

If an instruction operation has not yet been received (step S21: NO), the process remains at step S21 until such an operation is received. On the other hand, in the case that an instruction operation is received (step S21: YES), the process proceeds to the following step S22.

In step S22, the database processing unit 50 performs a searching process of the personality analysis information DB 72 in accordance with the instruction (selection of content) received in step S21, and reads out analysis data D2 that matches the search condition. For the search condition, there may be cited, for example, that the classification items of the events are identical or similar.

In step S23, as necessary, the analysis information editing unit 62 performs an editing process on the analysis data D2 that was read out in step S22. As such an editing process, there may be cited, for example, [1] an annotation process for annotating moving images or still images, [2] a graph creation process for expressing time series data in the form of a graph, and [3] a thinning-out process/trimming process for shortening the time required to reproduce the moving images.

Hereinafter, the analysis data D2 and the analysis content D4 may be collectively referred to as “personality analysis information” in certain cases. Moreover, the edited analysis content D4 may be recorded in the personality analysis information DB 72.

In step S24, the analysis information output unit 30 outputs to the user U the personality analysis information that was acquired in step S22 or step S23. Prior to the output thereof, the transmission/reception control unit 52 transmits the personality analysis information to the wearable computer 16. The wearable computer 16 visually displays the personality analysis information (in this instance, the analysis content D4) utilizing the display function that is incorporated therein.

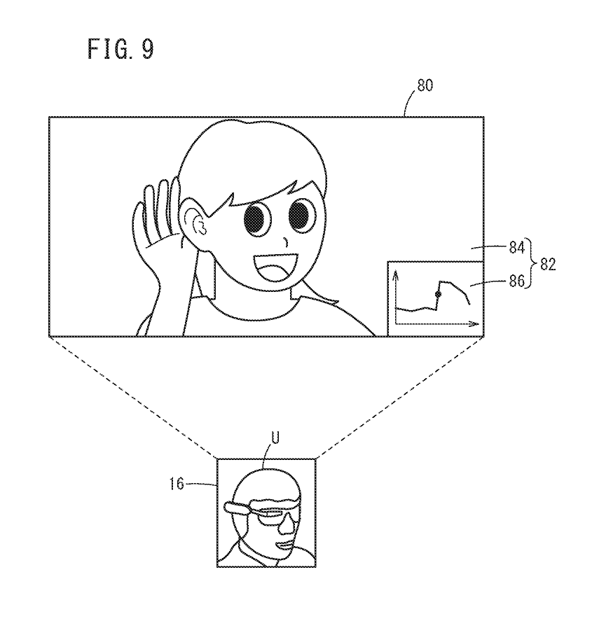

As shown in FIG. 9, within the display area 80 of the wearable computer 16, a virtual world 82 for displaying behaviors and actions from the eyes of the user U is generated. Such a virtual world 82 is composed of a reproduced moving image 84 showing a situation toward which the user U is facing and conducting a conversation, and a graph 86 superimposed on a lower right corner of the reproduced moving image 84. The graph 86 shows changes over time of the state (for example, the emotional state value E or the pulse rate) of the user U.

In this manner, the analysis information output unit 30 may be configured to be capable of generating the virtual world 82 in which a behavior of the user U is virtually reproduced using at least the featured log. In accordance with this feature, it is possible to reproduce experiences of the user U through the virtual world 82, and to enable the user U to look back from an objective standpoint on actions taken by the user himself.

For example, in the case of a date with one's girlfriend or boyfriend, it is possible to objectively grasp, in various events including eating a meal, driving, watching a movie, which scene the user U felt happiest in, and how the user himself or herself behaved or acted in the scene. Similarly, the user U can receive knowledge concerning a motive for quarreling, a motive for reconciling, or emotions at that time.

Also, when spending leisure time going for a stroll alone, it is possible to objectively grasp types of subjects the user himself or herself was interested in, what kind of means could be employed in order to encounter interesting subjects, and what kinds of behaviors were exhibited by the user when coming into contact with interesting subjects. In addition, in the case that the object of interest is a product, it is also possible to know what kind of emotional change accompanied the purchase of such a product, or what kind of emotional change accompanied the abstaining from purchasing the product.

In the case of the former (on a date), in addition to leaving a communication with one's significant companion as a memory, by looking back on such a communication, it becomes an impetus for the user to acquire knowledge of one's own characteristics or qualities when socializing. In the case of the latter (strolling alone), in addition to leaving unexpected new encounters as memories, by looking back on such encounters, it becomes an impetus for the user to make new discoveries concerning oneself, such as one's own potential desires or the like.

### <Fourth Operation: Output of Analysis Content D4 (Second Example)>

Incidentally, there may be cases in which the user U wants to discover an event or action that matches with his or her own characteristics (for example, personality or lifestyle). In this case, the life log utilization system 10 is capable of providing the analysis content D4 to the user U.

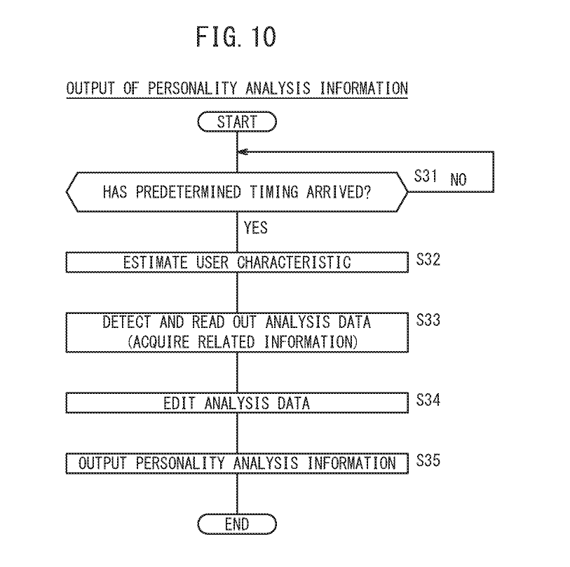

In step S31 of FIG. 10, the personality analysis support server 12 determines whether or not a predetermined timing has arrived. The predetermined timing may be a periodic timing (for example, every month), or may be an irregular timing (for example, a point in time when an instruction operation of the HMI by the user U is received).

In the case that the predetermined timing has not yet arrived (step S31: NO), the process remains at step S31 until arrival thereof. On the other hand, in the case that the predetermined timing has arrived (step S31: YES), the process proceeds to the following step S32.

In step S32, the characteristic estimating unit 64 estimates a characteristic of the user on the basis of analysis data D2 of the user U that was accumulated in the past. Prior to making the estimation, the characteristic estimating unit 64 acquires the user characteristic data D3 from the user characteristic DB 73 of the data center 14.

FIG. 11 is a diagram illustrating an example of a data structure of the user characteristic data D3. The present drawing corresponds to table data indicating a relationship of the user characteristic with respect to combinations of three types of classification items (emotion, event and behavior). For example, in the case that the user's emotion is “pleasure”, the event is “encounter with a person”, and the behavior is “talking to the person (engaged in conversation)”, a user characteristic of “active” is obtained. In the content of the user characteristics, in addition to active, there may be included, for example, steadiness, being emotional, short temper, gentleness, and economical.

By referring to the acquired user characteristic data D3, the characteristic estimating unit 64 estimates the user characteristic in accordance with a combination of the classification items included in the analysis data D2. For example, in the case there are plural sets of the analysis data D2, the most appropriate user characteristic can be determined using a statistical method including adoption of a mode value (majority decision) and average processing.

In this manner, the characteristic estimating unit 64 may estimate the characteristic of the user U by extracting a commonality in the event information or the behavior information included in a plurality of featured logs. In accordance with this feature, it is possible to express characteristics of the user that were performed unconsciously.

In step S33, the related information acquisition unit 66 acquires behavior information or event information (hereinafter referred to as related information) that is highly relevant to the user characteristic estimated in step S32. Prior to acquiring such information, the database processing unit 50 performs a search process on the data in the personality analysis information DB 72, and from among the analysis data D2 of other users (other target persons) sharing the same user characteristic, data is read out for which classification items of the events and behaviors are identical or similar.

Thereafter, the related information acquisition unit 66 acquires, as the aforementioned related information, a portion (for example, collected data) of the analysis data D2 that was read out by the database processing unit 50. Moreover, the related information is not limited to information (the analysis data D2) derived from the life log D1, but may be information obtained separately from an external network.

In step S34, as necessary, the analysis information editing unit 62 performs an editing process on the analysis data D2 (related information) that was acquired in step S33. As such an editing process, similar to the case of step S23, an annotation process, a graph creation process, a thinning-out process/trimming process may be performed.

In step S35, the analysis information output unit 30 outputs to the user U the personality analysis information that was acquired in step S33 or step S34. Prior to the output thereof, the transmission/reception control unit 52 transmits the personality analysis information to the wearable computer 16. The wearable computer 16 visually displays the analysis content D4 utilizing the display function that is incorporated therein.

In this manner, from among the related information acquired by the related information acquisition unit 66, the analysis information output unit 30 may extract information related to a behavior or an event that the user U has not yet experienced, and may output the extracted information at a predetermined timing. Consequently, it becomes possible to experience a behavior or an event in a pseudo manner by way of the related information, which enables the target person U to have a new awareness of himself.

### [Effects of the Life Log Utilization System 10]

As discussed above, the life log utilization system 10 includes [1] the event information acquisition unit 56 that acquires event information in relation to an event occurring in the vicinity of the user U (target person) or in relation to an event involving the user U, [2] the emotion information acquisition unit 58 that acquires emotion information indicative of the emotional state of the user U, [3] the behavior information acquisition unit 26 that acquires behavior information indicative of a behavior of the user U, [4] the life log DB 71 (life log recording unit) that associates the event information, the emotion information, and the behavior information, which are acquired at a same time or within a predetermined time period, and records the associated information as a life log D1 of the user U, and [5] the analysis information output unit 30 that outputs the recorded life log D1 or information (analysis data D2, analysis content D4) generated from the life log D1 as personality analysis information.

Further, the life log utilization method and program include [1] the event information acquisition step (S2) of acquiring the event information, [2] the emotion information acquisition step (S3) of acquiring the emotion information, [3] the behavior information acquisition step (S1a) of acquiring the behavior information, [4] the recording step (S4) of recording the life log D1 of the target person (U), and [5] the outputting step (S24, S35) of outputting the life log D1 or information (analysis data D2, analysis content D4) generated from the life log D1.

In the foregoing manner, the event information, the emotion information, and the behavior information are associated when the life log D1 is recorded, and such a life log D1 or information generated from the life log D1 is output. Therefore, from the standpoint of psychological behavior, it is possible to present useful information related to the personality of the user U, to the user U.

In particular, the life log utilization system 10 may further include [6] the biometric information acquisition unit 28 that acquires biometric information indicative of biological activity of the user U, and [7] the emotion information acquisition unit 58 may acquire the emotion information on the basis of the biometric information acquired by the biometric information acquisition unit 28.

### [Supplemental Considerations]

The present invention is not limited to the above-described embodiment, and it goes without saying that the present invention may be freely modified within a range that does not depart from the scope of the invention. Alternatively, the respective configurations may be arbitrarily combined within a range in which technical inconsistencies do not occur.

What is claimed is:

1. A life log utilization system comprising:
an event information acquisition unit configured to acquire event information in relation to an event occurring in a vicinity of a target person or in relation to an event involving the target person;
an emotion information acquisition unit configured to acquire emotion information indicative of an emotional state of the target person;
a behavior information acquisition unit configured to acquire behavior information indicative of a behavior of the target person;
a life log recording unit configured to associate the event information acquired by the event information acquisition unit, the emotion information acquired by the emotion information acquisition unit, and the behavior information acquired by the behavior information acquisition unit, which are acquired at a same time or within a predetermined time period, and to record the associated information as a life log of the target person; and
an analysis information output unit configured to output the life log recorded by the life log recording unit or information generated from the life log as personality analysis information.

2. The life log utilization system according to claim 1, further comprising:
a biometric information acquisition unit configured to acquire biometric information indicative of biological activity of the target person;
wherein the emotion information acquisition unit acquires the emotion information, based on the biometric information acquired by the biometric information acquisition unit.

3. The life log utilization system according to claim 1, further comprising:
a life log specifying unit configured to specify, as a featured log, the life log for which a change in the emotion information thereof is equal to or greater than a threshold value;
wherein the analysis information output unit outputs the personality analysis information in relation to the featured log specified by the life log specifying unit.

4. The life log utilization system according to claim 1, further comprising:
a life log specifying unit configured to specify, as a featured log, the life log for which a parameter in the life log or an indicator quantified or qualified using the parameter is specific, from among the life logs recorded in a past by the life log recording unit;
wherein the analysis information output unit outputs the personality analysis information in relation to the featured log specified by the life log specifying unit.

5. The life log utilization system according to claim 3, further comprising a characteristic estimating unit configured to estimate a characteristic of the target person by extracting a commonality in the event information or the behavior information included in a plurality of the featured logs.

6. The life log utilization system according to claim 4, further comprising a characteristic estimating unit configured to estimate a characteristic of the target person by extracting a commonality in the event information or the behavior information included in a plurality of the featured logs.

7. The life log utilization system according to claim 5, further comprising:
a related information acquisition unit configured to acquire related information which is made up of the behavior information or the event information having a high degree of relevance to the characteristic estimated by the characteristic estimating unit;
wherein the analysis information output unit extracts, from among the related information acquired by the related information acquisition unit, information that is related to a behavior or an event that the target person has not yet experienced, and outputs the extracted information at a predetermined timing to the target person.

8. The life log utilization system according to claim 6, further comprising:
a related information acquisition unit configured to acquire related information which is made up of the behavior information or the event information having a high degree of relevance to the characteristic estimated by the characteristic estimating unit;
wherein the analysis information output unit extracts, from among the related information acquired by the related information acquisition unit, information that is related to a behavior or an event that the target person has not yet experienced, and outputs the extracted information at a predetermined timing to the target person.

9. The life log utilization system according to claim 3, wherein the analysis information output unit is configured to generate a virtual world in which a behavior of the target person is virtually reproduced using at least the featured log.

10. The life log utilization system according to claim 4, wherein the analysis information output unit is configured to generate a virtual world in which a behavior of the target person is virtually reproduced using at least the featured log.

11. A life log utilization method configured to be executed by one or a plurality of computers, the life log utilization method comprising:
an event information acquisition step of acquiring event information in relation to an event occurring in a vicinity of a target person or in relation to an event involving the target person;
an emotion information acquisition step of acquiring emotion information indicative of an emotional state of the target person;
a behavior information acquisition step of acquiring behavior information indicative of a behavior of the target person;
a recording step of associating the event information, the emotion information, and the behavior information, which are acquired at a same time or within a predetermined time period, and recording the associated information as a life log of the target person; and
an outputting step of outputting the recorded life log or information generated from the life log as personality analysis information.

12. A non-transitory recording medium in which there is recorded a life log utilization program configured to cause one or a plurality of computers to perform:
an event information acquisition step of acquiring event information in relation to an event occurring in a vicinity of a target person or in relation to an event involving the target person;
an emotion information acquisition step of acquiring emotion information indicative of an emotional state of the target person;
a behavior information acquisition step of acquiring behavior information indicative of a behavior of the target person;
a recording step of associating the event information, the emotion information, and the behavior information, which are acquired at a same time or within a predetermined time period, and recording the associated information as a life log of the target person; and
an outputting step of outputting the recorded life log or information generated from the life log as personality analysis information.
